(19) United States
(12) Patent Application Publication
(10) Pub. No.: US 2019/0275499 A1
(43) Pub. Date: Sep. 12, 2019

(54) Sr-Ce-Yb-O Catalysts for Oxidative Coupling of Methane

(71) Applicants: Wugeng LIANG, Richmond, TX (US); Vidya Sagar Reddy SARSANI, Pearland, TX (US); David WEST, Bellaire, TX (US); Hector PEREZ, Angleton, TX (US); Aghaddin MAMEDOV, Sugar Land, TX (US); Sabic Global Technologies, B.V., BERGEN OP ZOOM (NL)

(72) Inventors: Wugeng LIANG, Sugar Land, TX (US); Vidya Sagar Reddy SARSANI, Sugar Land, TX (US); David WEST, Sugar Land, TX (US); Hector PEREZ, Sugar Land, TX (US); Aghaddin MAMEDOV, Sugar Land, TX (US)

(21) Appl. No.: 16/347,329

(22) PCT Filed: Nov. 7, 2017
(86) PCT No.: PCT/US2017/060371

§ 371 Date: May 3, 2019

Related U.S. Application Data
(60) Provisional application No. 62/418,480, filed on Nov. 7, 2016.

(51) Int. Cl. B01J 23/00; C07C 2/84; B01J 37/04; B01J 37/08

(52) U.S. Cl. CPC B01J 23/002; C07C 2/84; B01J 37/04; C07C 11/04; B01J 2523/24; B01J 2523/3712; B01J 2523/3787; B01J 37/08

(57) ABSTRACT

An oxidative coupling of methane (OCM) catalyst composition characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states. A method of making an oxidative coupling of methane (OCM) catalyst composition comprising (a) forming an oxide precursor mixture, wherein the oxide precursor mixture comprises one or more compounds comprising a Sr cation, one or more compounds comprising a Ce cation, and one or more compounds comprising a Yb cation, and wherein the oxide precursor mixture is characterized by a molar ratio of Sr:(Ce+Yb) that is not about 1:1, and (b) calcining at least a portion of the oxide precursor mixture to form the OCM catalyst composition, wherein the OCM catalyst composition comprises Sr—Ce—Yb—O perovskite in an amount of less than about 75.0 wt. %.

### CROSS REFERENCE TO RELATED APPLICATIONS

This application is a filing under 35 U.S.C. 371 of International Application No. PCT/US2017/060371 filed Nov. 7, 2017, entitled “Sr—Ce—Yb—O Catalysts for Oxidative Coupling of Methane” which claims priority to U.S. Provisional Application No. 62/418,480 filed Nov. 7, 2016, which applications are incorporated by reference herein in their entirety.

### TECHNICAL FIELD

The present disclosure relates to catalyst compositions for oxidative coupling of methane (OCM), more specifically catalyst compositions based on oxides of Sr, Ce and Yb for OCM, and methods of making and using same.

### BACKGROUND

Hydrocarbons, and specifically olefins such as ethylene, are typically building blocks used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Oxidative coupling of the methane (OCM) has been the target of intense scientific and commercial interest for more than thirty years due to the tremendous potential of such technology to reduce costs, energy, and environmental emissions in the production of ethylene (C₂H4). As an overall reaction, in the OCM, CH₄ and O₂ react exothermically over a catalyst to form C₂H4, water (H₂O) and heat.

Ethylene can be produced by OCM as represented by Equations (I) and (II):

2CH₄+O₂→C₂H₄+2H₂O ΔH=−67 kcal/mol (I)

2CH₄+½O₂→C₂H₆+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂O ΔH=−124 kcal/mol (III)

CH₄+2O₂→CO₂+2H₂O ΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored deep oxidation products.

Generally, in the OCM, CH₄ is first oxidatively converted into ethane (C₂H₆), and then into C₂H₄. CH₄ is activated heterogeneously on a catalyst surface, forming methyl free radicals (e.g., CH₃.), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with oxygen on the catalyst surface and/or in the gas phase, which produce (undesirable) carbon monoxide and carbon dioxide. Some of the best reported OCM outcomes encompass a ˜20% conversion of methane and ˜80% selectivity to desired C₂ hydrocarbons.

There are many catalyst systems developed for OCM processes, but such catalyst systems have many shortcomings. For example, conventional catalysts systems for OCM display catalyst performance problems, stemming from a need for high reaction temperatures. Thus, there is an ongoing need for the development of catalyst compositions for OCM processes.

### BRIEF SUMMARY

Disclosed herein is an oxidative coupling of methane (OCM) catalyst composition characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

Also disclosed herein is a method of making an oxidative coupling of methane (OCM) catalyst composition comprising (a) forming an oxide precursor mixture, wherein the oxide precursor mixture comprises one or more compounds comprising a Sr cation, one or more compounds comprising a Ce cation, and one or more compounds comprising a Yb cation, and wherein the oxide precursor mixture is characterized by a molar ratio of Sr:(Ce+Yb) that is not about 1:1, and (b) calcining at least a portion of the oxide precursor mixture to form the OCM catalyst composition, wherein the OCM catalyst composition comprises Sr—Ce—Yb—O perovskite in an amount of less than about 75.0 wt. %.

Further disclosed herein is a method for producing olefins comprising (a) introducing a reactant mixture to a reactor comprising an oxidative coupling of methane (OCM) catalyst composition, wherein the reactant mixture comprises methane (CH₄) and oxygen (O₂), wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states, (b) allowing at least a portion of the reactant mixture to contact at least a portion of the OCM catalyst composition and react via an OCM reaction to form a product mixture comprising olefins, (c) recovering at least a portion of the product mixture from the reactor, and (d) recovering at least a portion of the olefins from the product mixture.

### DETAILED DESCRIPTION

Disclosed herein are oxidative coupling of methane (OCM) catalyst compositions and methods of making and using same. In an aspect, an OCM catalyst composition can be characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states. The overall general formula Sr_{1.0}CeₐYb_{b}O_{c} further excludes the overall general formula SrCe₍₁₋ₓ₎YbₓO_{(3-x/2)}, wherein x is from about 0.01 to about 0.99.

A method of making an oxidative coupling of methane (OCM) catalyst composition can generally comprise the steps of (a) forming an oxide precursor mixture, wherein the oxide precursor mixture comprises one or more compounds comprising a Sr cation, one or more compounds comprising a Ce cation, and one or more compounds comprising a Yb cation, and wherein the oxide precursor mixture is characterized by a molar ratio of Sr:(Ce+Yb) that is not about 1:1; and (b) calcining at least a portion of the oxide precursor mixture to form the OCM catalyst composition, wherein the OCM catalyst composition comprises Sr—Ce—Yb—O perovskite in an amount of less than about 75.0 wt. %. The one or more compounds comprising a Sr cation can comprise Sr nitrate, Sr oxide, Sr hydroxide, Sr chloride, Sr acetate, Sr carbonate, or combinations thereof; the one or more compounds comprising a Ce cation can comprise Ce nitrate, Ce oxide, Ce hydroxide, Ce chloride, Ce acetate, Ce carbonate, or combinations thereof; and the one or more compounds comprising a Yb cation can comprise Yb nitrate, Yb oxide, Yb hydroxide, Yb chloride, Yb acetate, Yb carbonate, or combinations thereof.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

In an aspect, a method for producing olefins can comprise introducing a reactant mixture to a reactor comprising an oxidative coupling of methane (OCM) catalyst composition to form a product mixture comprising olefins, wherein the reactant mixture comprises methane (CH₄) and oxygen (O₂), and wherein the OCM catalyst composition can be characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

The reactant mixture can be a gaseous mixture. The reactant mixture can comprise a hydrocarbon or mixtures of hydrocarbons, and oxygen. In some aspects, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an aspect, the reactant mixture can comprise CH₄ and O₂.

The O₂ used in the reaction mixture can be oxygen gas (which may be obtained via a membrane separation process), technical oxygen (which may contain some air), air, oxygen enriched air, and the like, or combinations thereof.

The reactant mixture can further comprise a diluent. The diluent is inert with respect to the OCM reaction, e.g., the diluent does not participate in the OCM reaction. In an aspect, the diluent can comprise water, nitrogen, inert gases, and the like, or combinations thereof.

The diluent can provide for heat control of the OCM reaction, e.g., the diluent can act as a heat sink. Generally, an inert compound (e.g., a diluent) can absorb some of the heat produced in the exothermic OCM reaction, without degrading or participating in any reaction (OCM or other reaction), thereby providing for controlling a temperature inside the reactor.

The diluent can be present in the reactant mixture in an amount of from about 0.5% to about 80%, alternatively from about 5% to about 50%, or alternatively from about 10% to about 30%, based on the total volume of the reactant mixture.

A method for producing olefins can comprise introducing the reactant mixture to a reactor, wherein the reactor comprises the OCM catalyst composition. The reactor can comprise an adiabatic reactor, an autothermal reactor, an isothermal reactor, a tubular reactor, a cooled tubular reactor, a continuous flow reactor, a fixed bed reactor, a fluidized bed reactor, a moving bed reactor, and the like, or combinations thereof. In an aspect, the reactor can comprise a catalyst bed comprising the OCM catalyst composition.

The reaction mixture can be introduced to the reactor at a temperature of from about 150° C. to about 1,000° C., alternatively from about 225° C. to about 900° C., or alternatively from about 250° C. to about 800° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an aspect, the reaction mixture can be introduced to the reactor at a temperature effective to promote an OCM reaction.

The reactor can be characterized by a temperature of from about 400° C. to about 1,200° C., alternatively from about 500° C. to about 1,100° C., or alternatively from about 600° C. to about 1,000° C.

The reactor can be characterized by a pressure of from about ambient pressure (e.g., atmospheric pressure) to about 500 psig, alternatively from about ambient pressure to about 200 psig, or alternatively from about ambient pressure to about 150 psig. In an aspect, the method for producing olefins as disclosed herein can be carried out at ambient pressure.

The reactor can be characterized by a gas hourly space velocity (GHSV) of from about 500 h⁻¹ to about 10,000,000 h⁻¹, alternatively from about 500 h⁻¹ to about 1,000,000 h⁻¹, alternatively from about 500 h⁻¹ to about 500,000 h⁻¹, alternatively from about 1,000 h⁻¹ to about 500,000 h⁻¹, alternatively from about 1,500 h⁻¹ to about 500,000 h⁻¹, or alternatively from about 2,000 h⁻¹ to about 500,000 h⁻¹. Generally, the GHSV relates a reactant (e.g., reactant mixture) gas flow rate to a reactor volume. GHSV is usually measured at standard temperature and pressure.

The reactor can comprise an OCM catalyst composition characterized by the overall general formula Sr_{1.0}CeₐYb_{b}O_{c}; wherein a is from about 0.01 to about 2.0, alternatively from about 0.1 to about 1.9, or alternatively from about 0.2 to about 1.5; wherein b is from about 0.01 to about 2.0, alternatively from about 0.1 to about 1.9, or alternatively from about 0.2 to about 1.5; wherein the sum (a+b) is not 1.0; and wherein c balances the oxidation states. As will be appreciated by one of the skill in the art, and with the help of this disclosure, each of the Sr, Ce and Yb can have multiple oxidation states within the OCM catalyst composition, and as such c can have any suitable value that allows for the oxygen anions to balance all the cations. The OCM catalyst composition characterized by the overall general formula Sr_{1.0}CeₐYb_{b}O_{c} as disclosed herein can further satisfy the condition that a molar ratio of Sr:(Ce+Yb) of the OCM catalyst composition is not about 1:1 (e.g., the molar ratio of Sr:(Ce+Yb) of the OCM catalyst composition excludes the value of about 1:1).

In some aspects, the OCM catalyst composition can comprise Sr—Ce—Yb—O perovskite. The OCM catalyst composition can comprise Sr—Ce—Yb—O perovskite in an amount of less than about 75.0 wt. %, alternatively less than about 50.0 wt. %, alternatively less than about 25.0 wt. %, alternatively less than about 20.0 wt. %, alternatively less than about 10.0 wt. %, alternatively less than about 5.0 wt. %, alternatively less than about 4.0 wt. %, alternatively less than about 3.0 wt. %, alternatively less than about 2.0 wt. %, alternatively less than about 1.0 wt. %, alternatively less than about 0.1 wt. %, alternatively less than about 0.01 wt. %, or alternatively less than about 0.001 wt. % Sr—Ce—Yb—O perovskite, based on the total weight of the OCM catalyst composition. Generally, a perovskite refers to a compound having the same crystal structure as calcium titanate.

In other aspects, the OCM catalyst composition comprises no Sr—Ce—Yb—O perovskite, e.g., the OCM catalyst composition is substantially free of Sr—Ce—Yb—O perovskite.

In an aspect, the overall general formula Sr_{1.0}CeₐYb_{b}O_{c} as disclosed herein further excludes the overall general formula SrCe₍₁₋ₓ₎YbₓO_{(3-x/2)}, wherein x is from about 0.01 to about 0.99. As will be appreciated by one of skill in the art, and with the help of this disclosure, the overall general formula SrCe₍₁₋ₓ₎YbₓO_{(3-x/2)} further satisfies the condition of a molar ratio of Sr:(Ce+Yb) being about 1:1. Further, as will be appreciated by one of skill in the art, and with the help of this disclosure, a Sr—Ce—Yb—O perovskite can be characterized by the overall general formula SrCe₍₁₋ₓ₎YbₓO_{(3-x/2)}, wherein x is from about 0.01 to about 0.99.

In an aspect, the overall general formula Sr_{1.0}CeₐYb_{b}O_{c} as disclosed herein further excludes the overall general formula Sr_{1.0}Ce_{0.9}Yb_{0.1}O_{y}, wherein y balances the oxidation states. As will be appreciated by one of skill in the art, and with the help of this disclosure, the overall general formula Sr_{1.0}Ce_{0.9}Yb_{0.1}O_{y}, further satisfies the condition of a molar ratio of Sr:(Ce+Yb) being about 1:1. Further, as will be appreciated by one of skill in the art, and with the help of this disclosure, a Sr—Ce—Yb—O perovskite can be characterized by the overall general formula Sr_{1.0}Ce_{0.9}Yb_{0.1}O_{y}, wherein y balances the oxidation states. Further, as will be appreciated by one of the skill in the art, and with the help of this disclosure, each of the Sr, Ce and Yb can have multiple oxidation states within a composition characterized by the overall general formula Sr_{1.0}Ce_{0.9}Yb_{0.1}O_{y}, and as such y can have any suitable value that allows for the oxygen anions to balance all the cations.

In an aspect, the OCM catalyst composition can comprise one or more oxides of a metal selected from the group consisting of strontium (Sr), cerium (Ce), and ytterbium (Yb); wherein the one or more oxides comprises a single metal oxide, mixtures of single metal oxides, a mixed metal oxide, mixtures of mixed metal oxides, mixtures of single metal oxides and mixed metal oxides, or combinations thereof. The OCM catalyst composition can comprise the one or more oxides of a metal selected from the group consisting of Sr, Ce, and Yb in an amount of equal to or greater than about 25 wt. %, alternatively equal to or greater than about 50 wt. %, alternatively equal to or greater than about 75 wt. %, alternatively equal to or greater than about 80 wt. %, alternatively equal to or greater than about 90 wt. %, alternatively equal to or greater than about 95 wt. %, alternatively equal to or greater than about 99 wt. %, or alternatively equal to or greater than about 99.9 wt. % one or more oxides, based on the total weight of the OCM catalyst composition. In some aspects, the OCM catalyst composition can comprise, consist of, or consist essentially of one or more oxides of a metal selected from the group consisting of Sr, Ce, and Yb.

For purposes of the disclosure herein, in aspects where the OCM catalyst composition comprises Sr—Ce—Yb—O perovskite, the Sr—Ce—Yb—O perovskite of the OCM catalyst composition can be referred to as a “perovskite phase;” and the one or more oxides of the OCM catalyst composition can be referred to as an “oxide phase.” Without wishing to be limited by theory, the perovskite phase and the oxide phase have different physical and chemical properties, owing to having different crystal structures: the perovskite phase has a calcium titanate type of crystal structure, while the oxide phase has a crystal structure that is different than the calcium titanate type of crystal structure. The OCM catalyst composition can be regarded as a composite comprising the perovskite phase and the oxide phase, wherein the perovskite phase and the oxide phase can be interspersed. In some aspects, the OCM catalyst composition can comprise a continuous perovskite phase having a discontinuous oxide phase dispersed therein. In other aspects, the OCM catalyst composition can comprise a continuous oxide phase having a discontinuous perovskite phase dispersed therein. In yet other aspects, the OCM catalyst composition can comprise both a continuous perovskite phase and a continuous oxide phase, wherein the perovskite phase and the oxide phase contact each other. In still yet other aspects, the OCM catalyst composition can comprise regions of perovskite phase and regions of oxide phase, wherein at least a portion the regions of the perovskite phase contact at least a portion of the regions of the oxide phase. In still yet other aspects, the OCM catalyst composition can comprise a continuous oxide phase, wherein the OCM catalyst composition can be substantially free of a perovskite phase. As will be appreciated by one of skill in the art, and with the help of this disclosure, the amounts of each Sr—Ce—Yb—O perovskite and one or more oxides present in the OCM catalyst composition contribute to the distribution of the perovskite phase and the oxide phase within the OCM catalyst composition.

As will be appreciated by one of skill in the art, and with the help of this disclosure, and without wishing to be limited by theory, the OCM reaction is a multi-step reaction, wherein each step of the OCM reaction could benefit from specific OCM catalytic properties. For example, and without wishing to be limited by theory, an OCM catalyst should exhibit some degree of basicity to abstract a hydrogen from CH₄ to form hydroxyl groups [OH] on the OCM catalyst surface, as well as methyl radicals (CH₃.). Further, and without wishing to be limited by theory, an OCM catalyst should exhibit oxidative properties for the OCM catalyst to convert the hydroxyl groups [OH] from the catalyst surface to water, which can allow for the OCM reaction to continue (e.g., propagate). Further, as will be appreciated by one of skill in the art, and with the help of this disclosure, and without wishing to be limited by theory, an OCM catalyst could also benefit from properties like oxygen ion conductivity and proton conductivity, which properties can be critical for the OCM reaction to proceed at a very high rate (e.g., its highest possible rate). Further, as will be appreciated by one of skill in the art, and with the help of this disclosure, and without wishing to be limited by theory, an OCM catalyst with a single phase might not provide all the necessary properties for an optimum OCM reaction (e.g., best OCM reaction outcome) at the best level, and as such conducting an optimum OCM reaction may require an OCM catalyst with tailored multi phases, wherein the various different phases can have optimum properties for various OCM reaction steps, and wherein the various different phases can provide synergistically for achieving the best performance for the OCM catalyst in an OCM reaction.

Without wishing to be limited by theory, in addition to the amounts of each phase present in the OCM catalyst composition, the distribution of different phases in the catalyst composition is also important. For example, and without wishing to be limited by theory, a high activity phase (e.g., a phase containing CeO₂) could be dispersed and/or isolated in smaller fractions throughout the overall OCM catalyst composition in order to minimize and/or prevent deep oxidation reactions (e.g., CO₂ formation).

In an aspect, the one or more oxides of a metal selected from the group consisting of Sr, Ce, and Yb can comprise a single metal oxide, mixtures of single metal oxides, a mixed metal oxide, mixtures of mixed metal oxides, mixtures of both single metal oxides and mixed metal oxides, or combinations thereof.

Nonlimiting examples of the one or more oxides present in the OCM catalyst composition include CeO₂, CeYbO, Sr₂CeO₄, SrYb₂O₄, and the like, or combinations thereof. As will be appreciated by one of skill in the art, and with the help of this disclosure, a portion of the one or more oxides, in the presence of water, such as atmospheric moisture, can convert to hydroxides, and it is possible that the OCM catalyst composition will comprise some hydroxides, due to exposing the OCM catalyst composition comprising the one or more oxides to water (e.g., atmospheric moisture).

The single metal oxide comprises one metal cation selected from the group consisting of Sr, Ce, and Yb. A single metal oxide can be characterized by the general formula MₓO_{y}; wherein M is the metal cation selected from the group consisting of Sr, Ce, and Yb; and wherein x and y are integers from 1 to 7, alternatively from 1 to 5, or alternatively from 1 to 3. A single metal oxide contains one and only one metal cation. Nonlimiting examples of single metal oxides suitable for use in the OCM catalyst compositions of the present disclosure include CeO₂, Ce₂O₃, SrO, and Yb₂O₃.

In an aspect, mixtures of single metal oxides can comprise two or more different single metal oxides, wherein the two or more different single metal oxides have been mixed together to form the mixture of single metal oxides. Mixtures of single metal oxides can comprise two or more different single metal oxides, wherein each single metal oxide can be selected from the group consisting of CeO₂, Ce₂O₃, SrO, and Yb₂O₃. Nonlimiting examples of mixtures of single metal oxides suitable for use in the OCM catalyst compositions of the present disclosure include Yb₂O₃—CeO₂, Yb₂O₃—SrO, CeO₂—SrO, and the like, or combinations thereof.

The mixed metal oxide comprises two or more different metal cations, wherein each metal cation can be independently selected from the group consisting of Sr, Ce, and Yb. A mixed metal oxide can be characterized by the general formula M¹ₓ₁M²ₓ₂O_{y}; wherein M¹ and M² are metal cations; wherein each of the M¹ and M² can be independently selected from the group consisting of Sr, Ce, and Yb; and wherein x1, x2 and y are integers from 1 to 15, alternatively from 1 to 10, or alternatively from 1 to 7. In some aspects, M¹ and M² can be cations of different chemical elements, for example M¹ can be a Ce cation and M² can be a Sr cation. In other aspects, M¹ and M² can be different cations of the same chemical element, wherein M¹ and M² can have different oxidation states. Nonlimiting examples of mixed metal oxides suitable for use in the OCM catalyst compositions of the present disclosure include CeYbO, Sr₂CeO₄, SrYb₂O₄, and the like, or combinations thereof.

In an aspect, mixtures of mixed metal oxides can comprise two or more different mixed metal oxides, wherein the two or more different mixed metal oxides have been mixed together to form the mixture of mixed metal oxides. Mixtures of mixed metal oxides can comprise two or more different mixed metal oxides, such as CeYbO, Sr₂CeO₄, SrYb₂O₄, and the like, or combinations thereof.

In an aspect, mixtures of single metal oxides and mixed metal oxides can comprise at least one single metal oxide and at least one mixed metal oxide, wherein the at least one single metal oxide and the at least one mixed metal oxide have been mixed together to form the mixture of single metal oxides and mixed metal oxides. Mixtures of single metal oxides and mixed metal oxides can comprise at least one single metal oxide and at least one mixed metal oxide, such as CeO₂ and Sr₂CeO₄; CeO₂, CeYbO, and Sr₂CeO₄; CeO₂, CeYbO, Sr₂CeO₄, and SrYb₂O₄; and the like; or combinations thereof.

The OCM catalyst compositions suitable for use in the present disclosure can be supported OCM catalyst compositions and/or unsupported OCM catalyst compositions. In some aspects, the supported OCM catalyst compositions can comprise a support, wherein the support can be catalytically active (e.g., the support can catalyze an OCM reaction). In other aspects, the supported OCM catalyst compositions can comprise a support, wherein the support can be catalytically inactive (e.g., the support cannot catalyze an OCM reaction). In yet other aspects, the supported OCM catalyst compositions can comprise a catalytically active support and a catalytically inactive support. Nonlimiting examples of a support suitable for use in the present disclosure include MgO, Al₂O₃, SiO₂, ZrO₂, and the like, or combinations thereof. As will be appreciated by one of skill in the art, and with the help of this disclosure, the support can be purchased or can be prepared by using any suitable methodology, such as for example precipitation/co-precipitation, sol-gel techniques, templates/surface derivatized metal oxides synthesis, solid-state synthesis of mixed metal oxides, microemulsion techniques, solvothermal techniques, sonochemical techniques, combustion synthesis, etc.

In an aspect, the OCM catalyst composition can further comprise a support, wherein at least a portion of the OCM catalyst composition contacts, coats, is embedded in, is supported by, and/or is distributed throughout at least a portion of the support. In such aspect, the support can be in the form of powders, particles, pellets, monoliths, foams, honeycombs, and the like, or combinations thereof. Nonlimiting examples of support particle shapes include cylindrical, discoidal, spherical, tabular, ellipsoidal, equant, irregular, cubic, acicular, and the like, or combinations thereof.

In an aspect, the OCM catalyst composition can further comprise a porous support. As will be appreciated by one of skill in the art, and with the help of this disclosure, a porous material (e.g., support) can provide for an enhanced surface area of contact between the OCM catalyst composition and the reactant mixture, which in turn would result in a higher CH₄ conversion to CH₃.

The OCM catalyst composition can be made by using any suitable methodology. In an aspect, a method of making an OCM catalyst composition can comprise a step of forming an oxide precursor mixture, wherein the oxide precursor mixture comprises one or more compounds comprising a Sr cation, one or more compounds comprising a Ce cation, and one or more compounds comprising a Yb cation, and wherein the oxide precursor mixture is characterized by a molar ratio of Sr:(Ce+Yb) that is not about 1:1.

The one or more compounds comprising a Sr cation comprises Sr nitrate, Sr oxide, Sr hydroxide, Sr chloride, Sr acetate, Sr carbonate, and the like, or combinations thereof. The one or more compounds comprising a Ce cation comprises Ce nitrate, Ce oxide, Ce hydroxide, Ce chloride, Ce acetate, Ce carbonate, and the like, or combinations thereof. The one or more compounds comprising a Yb cation comprises Yb nitrate, Yb oxide, Yb hydroxide, Yb chloride, Yb acetate, Yb carbonate, and the like, or combinations thereof.

In an aspect, the step of forming the oxide precursor mixture can comprise solubilizing the one or more compounds comprising a Sr cation, one or more compounds comprising a Ce cation, and one or more compounds comprising a Yb cation in an aqueous medium to form an oxide precursor aqueous solution. The aqueous medium can be water, or an aqueous solution. The oxide precursor aqueous solution can be formed by dissolving the one or more compounds comprising a Sr cation, one or more compounds comprising a Ce cation, one or more compounds comprising a Yb cation, or combinations thereof, in water or any suitable aqueous medium. As will be appreciated by one of skill in the art, and with the help of this disclosure, the one or more compounds comprising a Sr cation, one or more compounds comprising a Ce cation, and one or more compounds comprising a Yb cation can be dissolved in an aqueous medium in any suitable order. In some aspects, the one or more compounds comprising a Sr cation, one or more compounds comprising a Ce cation, and one or more compounds comprising a Yb cation can be first mixed together and then dissolved in an aqueous medium.

The oxide precursor aqueous solution can be dried to form the oxide precursor mixture. In an aspect, at least a portion of the oxide precursor aqueous solution can be dried at a temperature of equal to or greater than about 75° C., alternatively of equal to or greater than about 100° C., or alternatively of equal to or greater than about 125° C., to yield the oxide precursor mixture. The oxide precursor aqueous solution can be dried for a time period of equal to or greater than about 4 hours, alternatively equal to or greater than about 8 hours, or alternatively equal to or greater than about 12 hours.

In an aspect, a method of making an OCM catalyst composition can comprise a step of calcining at least a portion of the oxide precursor mixture to form the OCM catalyst composition, wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states. The oxide precursor mixture can be calcined at a temperature of equal to or greater than about 650° C., alternatively equal to or greater than about 800° C., or alternatively equal to or greater than about 900° C., to yield the OCM catalyst composition. The oxide precursor mixture can be calcined for a time period of equal to or greater than about 2 hours, alternatively equal to or greater than about 4 hours, or alternatively equal to or greater than about 6 hours.

In some aspects, at least a portion of the oxide precursor mixture can be calcined in an oxidizing atmosphere (e.g., in an atmosphere comprising oxygen, for example in air) to form the OCM catalyst composition. Without wishing to be limited by theory, the oxygen in the OCM catalyst compositions characterized by the overall general formula Sr_{1.0}CeₐYb_{b}O_{c} can originate in the oxidizing atmosphere used for calcining the oxide precursor mixture. Further, without wishing to be limited by theory, the oxygen in the OCM catalyst compositions characterized by the overall general formula Sr_{1.0}CeₐYb_{b}O_{c} can originate in the one or more compounds comprising a Sr cation, one or more compounds comprising a Ce cation, and one or more compounds comprising a Yb cation, provided that at least one of these compounds comprises oxygen in its formula, as is the case with nitrates, oxides, hydroxides, acetates, carbonates, etc.

In some aspects, the method of making an OCM catalyst composition can further comprise contacting the OCM catalyst composition with a support to yield a supported catalyst (e.g., an OCM supported catalyst, an OCM supported catalyst composition, etc.).

In other aspects, the method of making an OCM catalyst composition can comprise forming the OCM catalyst composition in the presence of the support, such that the resulting OCM catalyst composition (after the calcining step) comprises the support.

In an aspect, a method for producing olefins can comprise allowing at least a portion of the reactant mixture to contact at least a portion of the OCM catalyst composition and react via an OCM reaction to form a product mixture comprising olefins.

The product mixture comprises coupling products, partial oxidation products (e.g., partial conversion products, such as CO, H₂, CO₂), and unreacted methane. The coupling products can comprise olefins (e.g., alkenes, characterized by a general formula CₙH₂ₙ) and paraffins (e.g., alkanes, characterized by a general formula CₙH₂ₙ₊₂).

The product mixture can comprise C₂₊ hydrocarbons, wherein the C₂₊ hydrocarbons can comprise C₂ hydrocarbons and C₃ hydrocarbons. In an aspect, the C₂₊ hydrocarbons can further comprise C₄ hydrocarbons (C₄s), such as for example butane, iso-butane, n-butane, butylene, etc. The C₂ hydrocarbons can comprise ethylene (C₂H₄) and ethane (C₂H₆). The C₂ hydrocarbons can further comprise acetylene (C₂H₂). The C₃ hydrocarbons can comprise propylene (C₃H₆) and propane (C₃H₈).

Reactant conversions (e.g., methane conversion, oxygen conversion, etc.) and selectivities to certain products (e.g., selectivity to C₂₊ hydrocarbons, selectivity to C₂ hydrocarbons, selectivity to ethylene, etc.) can be calculated as disclosed in more detail in the Examples section, for example such as described in equations (1)-(3).

In an aspect, equal to or greater than about 10 mol %, alternatively equal to or greater than about 30 mol %, or alternatively equal to or greater than about 50 mol % of the methane in the reactant mixture can be converted to C₂₊ hydrocarbons.

In an aspect, the OCM catalyst composition as disclosed herein can be characterized by a C₂₊ selectivity that is increased by equal to or greater than about 1%, alternatively equal to or greater than about 2.5%, or alternatively equal to or greater than about 5%, when compared to a C₂₊ selectivity of an otherwise similar OCM catalyst composition that is not characterized by the overall general formula Sr_{1.0}CeₐYb_{b}O_{c} wherein a is from about 0.01 to about 2.0, wherein b is from about 0.01 to about 2.0, wherein the sum (a+b) is not 1.0, and wherein c balances the oxidation states. Generally, a selectivity to a certain product refers to the amount of that particular product formed divided by the total amount of products formed.

In an aspect, the OCM catalyst composition as disclosed herein can be characterized by a C₂₊ yield that is increased by equal to or greater than about 5%, alternatively equal to or greater than about 10%, or alternatively equal to or greater than about 20%, when compared to a C₂₊ yield of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states. The yield with respect to C₂₊ hydrocarbons refers to the amount of C₂₊ hydrocarbons recovered from the product mixture (which can be expressed as volume, mass, moles, %, etc.).

In an aspect, a method for producing olefins can comprise recovering at least a portion of the product mixture from the reactor, wherein the product mixture can be collected as an outlet gas mixture from the reactor. In an aspect, a method for producing olefins can comprise recovering at least a portion of the C₂ hydrocarbons from the product mixture. The product mixture can comprise C₂₊ hydrocarbons (including olefins), unreacted methane, and optionally a diluent. The water produced from the OCM reaction and the water used as a diluent (if water diluent is used) can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In an aspect, at least a portion of the C₂₊ hydrocarbons can be separated (e.g., recovered) from the product mixture to yield recovered C₂₊ hydrocarbons. The C₂₊ hydrocarbons can be separated from the product mixture by using any suitable separation technique. In an aspect, at least a portion of the C₂₊ hydrocarbons can be separated from the product mixture by distillation (e.g., cryogenic distillation).

In an aspect, at least a portion of the recovered C₂₊ hydrocarbons can be used for ethylene production. In some aspects, at least a portion of ethylene can be separated from the product mixture (e.g., from the C₂₊ hydrocarbons, from the recovered C₂₊ hydrocarbons) to yield recovered ethylene and recovered hydrocarbons, by using any suitable separation technique (e.g., distillation). In other aspects, at least a portion of the recovered hydrocarbons (e.g., recovered C₂₊ hydrocarbons after olefin separation, such as separation of C₂H₄ and C₃H₆) can be converted to ethylene, for example by a conventional steam cracking process.

A method for producing olefins can comprise recovering at least a portion of the olefins from the product mixture. In an aspect, at least a portion of the olefins can be separated from the product mixture by distillation (e.g., cryogenic distillation). As will be appreciated by one of skill in the art, and with the help of this disclosure, the olefins are generally individually separated from their paraffin counterparts by distillation (e.g., cryogenic distillation). For example ethylene can be separated from ethane by distillation (e.g., cryogenic distillation). As another example, propylene can be separated from propane by distillation (e.g., cryogenic distillation).

In an aspect, at least a portion of the unreacted methane can be separated from the product mixture to yield recovered methane. Methane can be separated from the product mixture by using any suitable separation technique, such as for example distillation (e.g., cryogenic distillation). At least a portion of the recovered methane can be recycled to the reactant mixture.

In an aspect, an OCM catalyst composition can comprise (i) equal to or greater than about 25 wt. % one or more oxides of a metal selected from the group consisting of Sr, Ce, and Yb, wherein the one or more oxides comprises a single metal oxide, mixtures of single metal oxides, a mixed metal oxide, mixtures of mixed metal oxides, mixtures of single metal oxides and mixed metal oxides, and the like, or combinations thereof; and (ii) less than about 75 wt. % Sr—Ce—Yb—O perovskite (e.g., SrCeYbO₃ with perovskite structure). In such aspect, the OCM catalyst composition (i) can be characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; and wherein c balances the oxidation states; and (ii) can further satisfy the condition that a molar ratio of Sr:(Ce+Yb) of the OCM catalyst composition is not about 1:1.

In an aspect, an OCM catalyst composition can comprise (i) equal to or greater than about 90 wt. % one or more oxides of a metal selected from the group consisting of Sr, Ce, and Yb, wherein the one or more oxides comprises CeO₂, CeYbO, Sr₂CeO₄, SrYb₂O₄, and the like, or combinations thereof; and (ii) less than about 10 wt. % Sr—Ce—Yb—O perovskite (e.g., SrCeYbO₃ with perovskite structure). In such aspect, the OCM catalyst composition can be characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; wherein c balances the oxidation states; and wherein the overall general formula Sr_{1.0}CeₐYb_{b}O_{c} further excludes the overall general formula SrCe₍₁₋ₓ₎YbₓO_{3-x/2)}, wherein x is from about 0.01 to about 0.99.

In an aspect, a method of making an OCM catalyst composition can comprise the steps of (a) forming a oxide precursor aqueous solution comprising Sr nitrate, Ce nitrate, and Yb nitrate, wherein the oxide precursor aqueous solution is characterized by a molar ratio of Sr:(Ce+Yb) that is not about 1:1; (b) drying at least a portion of the oxide precursor aqueous solution at a temperature of about 125° C. for about 12-18 h to form an oxide precursor mixture; and (c) calcining at least a portion of the oxide precursor mixture at a temperature of about 900° C. for about 4-8 h, for example in an oxidizing atmosphere, to form the OCM catalyst composition, wherein the OCM catalyst composition comprises (i) equal to or greater than about 25 wt. % one or more oxides of a metal selected from the group consisting of Sr, Ce, and Yb, wherein the one or more oxides comprises a single metal oxide, mixtures of single metal oxides, a mixed metal oxide, mixtures of mixed metal oxides, mixtures of single metal oxides and mixed metal oxides, and the like, or combinations thereof; and (ii) less than about 75 wt. % Sr—Ce—Yb—O perovskite (e.g., SrCeYbO₃ with perovskite structure).

In an aspect, a method for producing ethylene can comprise the steps of (a) introducing a reactant mixture to a reactor comprising an oxidative coupling of methane (OCM) catalyst composition, wherein the reactant mixture comprises methane (CH₄) and oxygen (O₂), wherein the OCM catalyst composition is characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; and wherein c balances the oxidation states; (b) allowing at least a portion of the reactant mixture to contact at least a portion of the OCM catalyst composition and react via an OCM reaction to form a product mixture comprising olefins, wherein the olefins comprise ethylene; (c) recovering at least a portion of the product mixture from the reactor, and (d) recovering at least a portion of the ethylene from the product mixture.

In an aspect, the OCM catalyst compositions characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; and wherein c balances the oxidation states, and methods of making and using same, as disclosed herein can advantageously display improvements in one or more composition characteristics when compared to an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

The OCM catalyst compositions characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; and wherein c balances the oxidation states, can display improved selectivity and yield when compared to the selectivity and yield of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

In an aspect, the composition of OCM catalyst compositions characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; and wherein c balances the oxidation states, as disclosed herein can be advantageously adjusted as necessary, based on the needs of the OCM reaction, to meet target criteria, such as a target selectivity and/or a target conversion, owing to a broader range of Sr, Ce and Yb content; and as such the OCM catalyst compositions as disclosed herein can display better performance when compared to otherwise similar OCM catalyst compositions having the sum (a+b) equal to 1.0. Additional advantages of the OCM catalyst compositions characterized by the overall general formula Sr_{1.0}CeₐYb_{b}O_{c}; wherein a is from about 0.01 to about 2.0; wherein b is from about 0.01 to about 2.0; wherein the sum (a+b) is not 1.0; and wherein c balances the oxidation states, and methods of making and using same, as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

Oxidative coupling of methane (OCM) catalyst compositions were prepared as follows.

A reference catalyst composition following the overall general formula Sr_{1.0}Ce_{0.9}Yb_{0.1}O was prepared as follows. 4.23 g of Sr(NO₃)₂, 7.82 g of Ce(NO₃)₃×6H₂O and 0.90 g of Yb(NO₃)₃×5H₂O were added into 25 ml deionized (DI) water to provide a mixture, which mixture was further agitated until all solids were dissolved and a clear solution was obtained. The obtained clear solution was dried at 125° C. overnight to produce a dried Sr—Ce—Yb—O precursor mixture. The dried Sr—Ce—Yb—O precursor mixture was calcined under air flow at 900° C. for 6 hours to produce the reference catalyst (e.g., Sr_{1.0}Ce_{0.9}Yb_{0.1}O catalyst). As will be appreciated by one of skill in the art, and with the help of this disclosure, the reference catalyst (e.g., Sr_{1.0}Ce_{0.9}Yb_{0.1}O catalyst) further satisfies the condition of a molar ratio of Sr:(Ce+Yb) being about 1:1. The reference catalyst follows the overall general formula SrCe₍₁₋ₓ₎YbₓO_{(3-x/2)}, wherein x is 0.1.

Catalyst #1 was prepared by following the same method as for the reference catalyst, but with an increased Yb amount used to yield the overall general formula Sr_{1.0}Ce_{0.9}Yb_{0.2}O. As will be appreciated by one of skill in the art, and with the help of this disclosure, catalyst #1 further satisfies the condition that a molar ratio of Sr:(Ce+Yb) of the catalyst #1 is not about 1:1.

Catalyst #2 was prepared by following the same method as for the reference catalyst, but with a further increased Yb amount used to yield the overall general formula Sr_{1.0}Ce_{0.9}Yb_{0.5}O. As will be appreciated by one of skill in the art, and with the help of this disclosure, catalyst #2 further satisfies the condition that a molar ratio of Sr:(Ce+Yb) of the catalyst #2 is not about 1:1.

Catalyst #3 was prepared by following the same method as for the reference catalyst, but with an increased Ce amount used to yield the overall general formula Sr_{1.0}Ce_{1.0}Yb_{0.1}O. As will be appreciated by one of skill in the art, and with the help of this disclosure, catalyst #3 further satisfies the condition that a molar ratio of Sr:(Ce+Yb) of the catalyst #3 is not about 1:1.

Catalyst #4 was prepared by following the same method as for the reference catalyst, but with a further increased Ce amount used to yield the overall general formula Sr_{1.0}Ce_{1.2}Yb_{0.1}O. As will be appreciated by one of skill in the art, and with the help of this disclosure, catalyst #4 further satisfies the condition that a molar ratio of Sr:(Ce+Yb) of the catalyst #4 is not about 1:1.

Catalyst #5 was prepared by following the same method as for the reference catalyst, but with increased Ce and Yb amounts used to yield the overall general formula Sr_{1.0}Ce_{1.0}Yb_{1.0}O. As will be appreciated by one of skill in the art, and with the help of this disclosure, catalyst #5 further satisfies the condition that a molar ratio of Sr:(Ce+Yb) of the catalyst #5 is not about 1:1.

### Example 2

The performance of the OCM catalyst compositions prepared as described in Example 1 was investigated.

OCM reactions were conducted by using catalysts prepared as described in Example 1 as follows. A mixture of methane and oxygen along with an internal standard, an inert gas (neon) were fed to a quartz reactor with an internal diameter (I.D.) of 2.3 mm heated by traditional clamshell furnace. A catalyst (e.g., catalyst bed) loading was 20 mg, and total flow rate of reactants was 40 standard cubic centimeters per minute (sccm). The reactor was first heated to a desired temperature under an inert gas flow and then a desired gas mixture was fed to the reactor. All OCM reactions were conducted at a methane to oxygen (CH₄:O₂) molar ratio of 7.4 and at a reactor temperature of 750° C. The products obtained from the OCM reaction were analyzed by using an online Agilent 6890 gas chromatograph (GC) with a thermal conductivity detector (TCD) and a flame ionization detector (FID).

Methane conversion was calculated according to equation (1). Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For example, the methane conversion can be calculated by using equation (1):

$${CH}_{4} conversion = \frac{{C}_{{CH}_{4}}^{in} - {C}_{{CH}_{4}}^{out}}{{C}_{{CH}_{4}}^{in}} \times 100 \% \qquad ( 1 )$$

wherein C_{CH}_{₄}ⁱⁿ=number of moles of C from CH₄ that entered the reactor as part of the reactant mixture; and C_{CH}_{₄}^{out}=number of moles of C from CH₄ that was recovered from the reactor as part of CH₄ the product mixture.

The oxygen conversion can be calculated by using equation (2):

$${O}_{2} conversion = \frac{{O}_{2}^{in} - {O}_{2}^{out}}{{O}_{2}^{in}} \times 100 \% \qquad ( 2 )$$

wherein O₂ⁱⁿ=number of moles of O₂ that entered the reactor as part of the reactant mixture; and O₂^{out}=number of moles of O₂ that was recovered from the reactor as part of the product mixture.

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a C, selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{CH8}=number of moles of C from CH₄ that were converted into C₃H₈; C_{C4s}=number of moles of C from CH₄ that were converted into C₄ hydrocarbons (C₄s); C_{CO2}=number of moles of C from CH₄ that were converted into CO₂; C_{CO}=number of moles of C from CH₄ that were converted into CO; etc.

A C₂₊ selectivity (e.g., selectivity to C₂₊ hydrocarbons) refers to how much C₂H₄, C₃H₆, C₂H₂, C₂H₆, C₃H₈, and C₄s were formed divided by the total products formed, including C₂H₄, C₃H₆, C₂H₂, C₂H₆, C₃H₈, C₄s, CO₂ and CO. For example, the C₂₊ selectivity can be calculated by using equation (3):

$${C}_{2 +} selectivity = \frac{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4 s}}\end{matrix}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4 s}} + {C}_{{CO}_{2}} + {C}_{CO}\end{matrix}} \times 100 \% \qquad ( 3 )$$

As will be appreciated by one of skill in the art, if a specific product and/or hydrocarbon product is not produced in a certain OCM reaction/process, then the corresponding C_{Cx} is 0, and the term is simply removed from selectivity calculations.

Further, a C₂₊ yield can be calculated as the product of C₂₊ selectivity and methane conversion, for example by using equation (4):

C₂₊ yield=methane conversion×C₂₊ selectivity (4)

For example, if a certain OCM reaction/process is characterized by a 50% methane conversion, and by a 50% C₂₊ selectivity, the resulting C₂₊ yield can be calculated as being 25% (=50%×50%).

The performance differences between the catalysts are demonstrated in Tables 1-3.

TABLE 1

| | | | | | C2+ |
|---|---|---|---|---|---|
| | | CH4 Conversion | O2 Conversion | C2+ Selectivity | Yield |
| | Catalyst composition | [%] | [%] | [%] | [%] |
| Reference | Sr1.0Ce0.9Yb0.1O | 18.0 | 92.8 | 77.8 | 14.0 |
| Catalyst | Following the | | | | |
| | SrCe₍₁₋ₓ₎YbₓO_{(3-x/2)} | | | | |
| | formula | | | | |
| Catalyst #1 | Sr1.0Ce0.9Yb0.2O | 19.1 | 94.8 | 78.8 | 15.1 |
| | Not following the | | | | |
| | SrCe₍₁₋ₓ₎YbₓO_{(3-x/2)} | | | | |
| | formula | | | | |
| Catalyst #2 | Sr1.0Ce0.9Yb0.5O | 19.1 | 91.0 | 78.8 | 15.1 |
| | Not following the | | | | |
| | SrCe₍₁₋ₓ₎YbₓO_{(3-x/2)} | | | | |
| | formula | | | | |

TABLE 2

| | | CH4 | | C2+ | |
|---|---|---|---|---|---|
| | Catalyst | Conversion | O2 Conversion | Selectivity | C2+ Yield |
| | Composition | [%] | [%] | [%] | [%] |
| Reference | Sr1.0Ce0.9Yb0.1O | 18.0 | 92.8 | 77.8 | 14.0 |
| Catalyst | Following the | | | | |
| | SrCe₍₁₋ₓ₎YbₓO_{(3-x/2)} | | | | |
| | formula | | | | |
| Catalyst #3 | Sr1.0Ce1.0Yb0.1O | 18.2 | 95.8 | 78.7 | 14.3 |
| | Not following the | | | | |
| | SrCe₍₁₋ₓ₎YbₓO_{(3-x/2)} | | | | |
| | formula | | | | |
| Catalyst #4 | Sr1.0Ce1.2Yb0.1O | 19.8 | 99.6 | 80.3 | 15.9 |
| | Not following the | | | | |
| | SrCe₍₁₋ₓ₎YbₓO_{(3-x/2)} | | | | |
| | formula | | | | |

TABLE 3

| | | CH4 | | C2+ | |
|---|---|---|---|---|---|
| | Catalyst | Conversion | O2 Conversion | Selectivity | C2+ Yield |
| | Composition | [%] | [%] | [%] | [%] |
| Reference | Sr1.0Ce0.9Yb0.1O | 18.0 | 92.8 | 77.8 | 14.0 |
| Catalyst | Following the | | | | |
| | SrCe₍₁₋ₓ₎YbₓO_{(3-x/2)} formula | | | | |
| Catalyst #5 | Sr1.0Ce1.0Yb1.0O | 18.5 | 99.3 | 74.5 | 13.8 |
| | Not following the | | | | |
| | SrCe₍₁₋ₓ₎YbₓO_{(3-x/2)} formula | | | | |

TABLE 4

| | | | | | | C₂₊ |
|---|---|---|---|---|---|---|
| | | | CH₄ | CH₄ | C₂₊ | Produc- |
| | Temper- | | flowrate | Conver- | Selec- | tivity |
| | ature | Catalyst | (ml/ | sion | tivity | (cc/ |
| Catalyst | (° C.) | loading | min) | (%) | (%) | min/g) |
| Reference | 725 | 20 mg | 52.3 | 19.8 | 79.8 | 411.2 |
| Catalyst | | | | | | |
| Catalyst #4 | 725 | 20 mg | 52.3 | 20.4 | 80.9 | 429.5 |
| Literature (1) | 750 | 600 mg | 3.3 | 52.6 | 60.1 | 1.74 |
| Literature (2) | 775 | 500 mg | 40.0 | 20.0 | 60.0 | 9.6 |

In Table 1, catalysts #1 and #2 have a higher Yb content, without an enhanced Ce content, when compared to the reference catalyst. Catalysts #1 and #2 show better performance as compared to the reference catalyst.

In Table 2, catalysts #3 and #4 have a higher Ce content, when compared to the reference catalyst. Catalyst #3 shows better performance as compared to the reference catalyst. Catalyst #4 clearly shows higher activity and higher selectivity as compared to the reference catalyst.

In Table 3, catalyst #5 has a higher Yb content and a higher Ce content, when compared to the reference catalyst. Catalyst #5 shows higher activity, but lower selectivity as compared to the reference catalyst.

The performance of the reference catalyst and of catalyst #4 was further compared to data available in the literature: J. Chem. Soc., Chem. Commun., 1987, p. 1639 (Literature (1); and J. Chem. Soc. Faraday Trans., 91 (1995), p. 1179 (Literature (2)), each of which is incorporated by reference herein in its entirety. The results of the comparison are displayed in Table 4. The data in Table 4 were collected as described for Tables 1-3, except for the flow rate, which was 60 sccm for the data in Table 4. The productivity with respect to C₂₊ hydrocarbons refers to the amount of C₂₊ hydrocarbons recovered from the product mixture (which can be expressed as volume, mass, moles, etc.) per unit of time (e.g., hours, minutes, seconds, etc.) per amount of catalyst used (e.g., g, kg, lb, etc.). The productivity with respect to a certain catalyst is a measure of effectiveness for that particular catalyst. The C₂₊ productivity of each catalyst was calculated as the C₂₊ formed (cc/min) over the same amount of the catalyst. The literature catalysts are Sr—Ce—Yb—O catalysts with pure perovskite structure, and as such the data in Table 4 indicate the superior performance of the reference catalyst comprising other oxides in addition to the perovskite oxides, as well as the superior performance of catalyst #4, as compared to the literature catalysts. The data in Tables 1-4 further confirm that a catalyst having tailored multi phases with required properties (e.g., reference catalyst, catalysts #1, #2, #3, #4, and #5) will perform better than a catalyst having a single phase alone. Since the reference catalyst performs better than the literature catalysts, any other catalysts performing better than the reference catalyst (e.g., catalysts #1, #2, #3, #4, and #5) will perform better than the literature catalysts.

Based on the above results in Tables 1-4, it is clearly demonstrated that a high performance OCM catalyst can be obtained for oxides of Sr, Ce, and Yb, without following overall general formula SrCe₍ₗ₋ₓ₎YbₓO_{(3-x/2)}. These data open up a new range of compositions of oxides of Sr, Ce, and Yb as catalysts for oxidative coupling of methane.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### Additional Disclosure

A first aspect, which is an oxidative coupling of methane (OCM) catalyst composition characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

A second aspect, which is the OCM catalyst composition of the first aspect, wherein the overall general formula Sr_{1.0}CeₐYb_{b}O_{c} further excludes the overall general formula SrCe₍₁₋ₓ₎YbₓO_{(3-x/2)}, wherein x is from about 0.01 to about 0.99.

A third aspect, which is the OCM catalyst composition of any one of the first and the second aspects, wherein the overall general formula Sr_{1.0}CeₐYb_{b}O_{c} further excludes the overall general formula Sr_{1.0}Ce_{0.9}Yb_{0.1}O_{y}, wherein y balances the oxidation states.

A fourth aspect, which is the OCM catalyst composition of any one of the first through the third aspects comprising less than about 75.0 wt. % Sr—Ce—Yb—O perovskite.

A fifth aspect, which is the OCM catalyst composition of any one of the first through the fourth aspects comprising one or more oxides of a metal selected from the group consisting of strontium (Sr), cerium (Ce), and ytterbium (Yb); wherein the one or more oxides comprises a single metal oxide, mixtures of single metal oxides, a mixed metal oxide, mixtures of mixed metal oxides, mixtures of single metal oxides and mixed metal oxides, or combinations thereof.

A sixth aspect, which is the OCM catalyst composition of the fifth aspect, wherein the one or more oxides are present in the OCM catalyst composition in an amount of equal to or greater than about 25 wt. %.

A seventh aspect, which is the OCM catalyst composition of any one of the first through the sixth aspects, wherein the one or more oxides comprise CeO₂, CeYbO, Sr₂CeO₄, SrYb₂O₄, or combinations thereof.

An eighth aspect, which is the OCM catalyst composition of any one of the first through the seventh aspects, wherein the single metal oxide comprises one metal cation selected from the group consisting of Sr, Ce, and Yb.

A ninth aspect, which is the OCM catalyst composition of any one of the first through the eighth aspects, wherein the single metal oxide comprises CeO₂.

A tenth aspect, which is the OCM catalyst composition of any one of the first through the ninth aspects, wherein the mixed metal oxide comprises two or more different metal cations, wherein each metal cation can be independently selected from the group consisting of Sr, Ce, and Yb.

An eleventh aspect, which is the OCM catalyst composition of any one of the first through the tenth aspects, wherein the mixed metal oxide comprises CeYbO, Sr₂CeO₄, SrYb₂O₄, or combinations thereof.

A twelfth aspect, which is the OCM catalyst composition of any one of the first through the eleventh aspects further comprising a support, wherein at least a portion of the OCM catalyst composition contacts, coats, is embedded in, is supported by, and/or is distributed throughout at least a portion of the support; wherein the support comprises MgO, Al₂O₃, SiO₂, ZrO₂, or combinations thereof; and wherein the support is in the form of particles, pellets, monoliths, foams, honeycombs, or combinations thereof.

A thirteenth aspect, which is the OCM catalyst composition of any one of the first through the twelfth aspects, wherein the OCM catalyst composition is characterized by a C₂₊ selectivity that is increased by equal to or greater than about 1%, when compared to a C₂₊ selectivity of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

A fourteenth aspect, which is the OCM catalyst composition of any one of the first through the thirteenth aspects, wherein the OCM catalyst composition is characterized by a C₂₊ yield that is increased by equal to or greater than about 5%, when compared to a C₂₊ yield of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

A fifteenth aspect, which is a method of making an oxidative coupling of methane (OCM) catalyst composition comprising (a) forming an oxide precursor mixture, wherein the oxide precursor mixture comprises one or more compounds comprising a Sr cation, one or more compounds comprising a Ce cation, and one or more compounds comprising a Yb cation, and wherein the oxide precursor mixture is characterized by a molar ratio of Sr:(Ce+Yb) that is not about 1:1; and (b) calcining at least a portion of the oxide precursor mixture to form the OCM catalyst composition, wherein the OCM catalyst composition comprises Sr—Ce—Yb—O perovskite in an amount of less than about 75.0 wt. %.

A sixteenth aspect, which is the method of the fifteenth aspect, wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

A seventeenth aspect, which is the method of any one of the fifteenth and the sixteenth aspects, wherein the step (a) of forming an oxide precursor mixture further comprises (i) solubilizing the one or more compounds comprising a Sr cation, one or more compounds comprising a Ce cation, and one or more compounds comprising a Yb cation in an aqueous medium to form an oxide precursor aqueous solution; and (ii) drying at least a portion of the oxide precursor aqueous solution to form the oxide precursor mixture.

An eighteenth aspect, which is the method of the seventeenth aspect, wherein the oxide precursor aqueous solution is dried at a temperature of equal to or greater than about 75° C.

A nineteenth aspect, which is the method of any one of the fifteenth through the eighteenth aspects, wherein the oxide precursor mixture is calcined at a temperature of equal to or greater than about 650° C.

A twentieth aspect, which is the method of any one of the fifteenth through the nineteenth aspects, wherein the one or more compounds comprising a Sr cation comprises Sr nitrate, Sr oxide, Sr hydroxide, Sr chloride, Sr acetate, Sr carbonate, or combinations thereof; wherein the one or more compounds comprising a Ce cation comprises Ce nitrate, Ce oxide, Ce hydroxide, Ce chloride, Ce acetate, Ce carbonate, or combinations thereof; and wherein the one or more compounds comprising a Yb cation comprises Yb nitrate, Yb oxide, Yb hydroxide, Yb chloride, Yb acetate, Yb carbonate, or combinations thereof.

A twenty-first aspect, which is an OCM catalyst produced by the method of any one of the fifteenth through the twentieth aspects.

A twenty-second aspect, which is a method of making an oxidative coupling of methane (OCM) catalyst composition comprising (a) forming an oxide precursor aqueous solution comprising Sr nitrate, Ce nitrate, and Yb nitrate, wherein the oxide precursor aqueous solution is characterized by a molar ratio of Sr:(Ce+Yb) that is not about 1:1; (b) drying at least a portion of the oxide precursor aqueous solution at a temperature of equal to or greater than about 75° C. to form an oxide precursor mixture; and (c) calcining at least a portion of the oxide precursor mixture at a temperature of equal to or greater than about 650° C. to form the OCM catalyst composition, wherein the OCM catalyst composition comprises a Sr—Ce—Yb—O perovskite in an amount of less than about 75.0 wt. %.

A twenty-third aspect, which is the method of the twenty-second aspect, wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

A twenty-fourth aspect, which is an oxidative coupling of methane (OCM) catalyst composition produced by (a) solubilizing one or more compounds comprising a Sr cation, one or more compounds comprising a Ce cation, and one or more compounds comprising a Yb cation in an aqueous medium to form an oxide precursor aqueous solution, wherein the oxide precursor aqueous solution is characterized by a molar ratio of Sr:(Ce+Yb) that is not about 1:1; (b) drying at least a portion of the oxide precursor aqueous solution at a temperature of equal to or greater than about 75° C. to form the oxide precursor mixture; and (c) calcining at least a portion of the oxide precursor mixture at a temperature of equal to or greater than about 650° C. to form the OCM catalyst composition, wherein the OCM catalyst composition comprises a Sr—Ce—Yb—O perovskite in an amount of less than about 75.0 wt. %.

A twenty-fifth aspect, which is the OCM catalyst composition of the twenty-fourth aspect having the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

A twenty-sixth aspect, which is a method for producing olefins comprising (a) introducing a reactant mixture to a reactor comprising an oxidative coupling of methane (OCM) catalyst composition, wherein the reactant mixture comprises methane (CH₄) and oxygen (O₂), wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states; (b) allowing at least a portion of the reactant mixture to contact at least a portion of the OCM catalyst composition and react via an OCM reaction to form a product mixture comprising olefins; (c) recovering at least a portion of the product mixture from the reactor, and (d) recovering at least a portion of the olefins from the product mixture.

A twenty-seventh aspect, which is the method of the twenty-sixth aspect, wherein the OCM catalyst composition is characterized by a C₂₊ selectivity that is increased by equal to or greater than about 1%, when compared to a C₂₊ selectivity of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states; and wherein the OCM catalyst composition is characterized by a C₂₊ yield that is increased by equal to or greater than about 5%, when compared to a C₂₊ yield of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. An oxidative coupling of methane (OCM) catalyst composition characterized by the overall general formula Sr_{1.0}CeₐY_{b}O_{c}, wherein a is from about 0.01 to about 2.0, wherein b is from about 0.01 to about 2.0, wherein the sum (a+b) is not 1.0, and wherein c balances the oxidation states.

2. The OCM catalyst composition of claim 1, wherein the overall general formula Sr_{1.0}CeₐY_{b} O_{c} further excludes the overall general formula SrCe₍₁₋ₓ₎YbₓO_{(3-x/2)}, wherein x is from about 0.01 to about 0.99.

3. The OCM catalyst composition of claim 1, wherein the overall general formula Sr_{1.0}CeₐYb_{b}O_{c} further excludes the overall general formula Sr_{1.0}Ce_{0.9}Yb_{0.1}O_{y}, wherein y balances the oxidation states.

4. The OCM catalyst composition of claim 1 comprising less than about 75.0 wt. % Sr—Ce—Yb—O perovskite.

5. The OCM catalyst composition of claim 1 comprising one or more oxides of a metal selected from the group consisting of strontium (Sr), cerium (Ce), and ytterbium (Yb); wherein the one or more oxides comprises a single metal oxide, mixtures of single metal oxides, a mixed metal oxide, mixtures of mixed metal oxides, mixtures of single metal oxides and mixed metal oxides, or combinations thereof.

6. The OCM catalyst composition of claim 5, wherein the one or more oxides are present in the OCM catalyst composition in an amount of equal to or greater than about 25 wt. %.

7. The OCM catalyst composition of claim 1, wherein the one or more oxides comprise CeO₂, CeYbO, Sr₂CeO₄, SrYb₂O₄, or combinations thereof.

8. The OCM catalyst composition of claim 1, wherein the single metal oxide comprises one metal cation selected from the group consisting of Sr, Ce, and Yb.

9. The OCM catalyst composition of claim 1, wherein the single metal oxide comprises CeO₂.

10. The OCM catalyst composition of claim 1, wherein the mixed metal oxide comprises two or more different metal cations, wherein each metal cation can be independently selected from the group consisting of Sr, Ce, and Yb.

11. The OCM catalyst composition of claim 1, wherein the mixed metal oxide comprises CeYbO, Sr₂CeO₄, SrYb₂O₄, or combinations thereof.

12. The OCM catalyst composition of claim 1, wherein the OCM catalyst composition is characterized by (1) a C₂₊ selectivity that is increased by equal to or greater than about 1%, when compared to a C₂₊ selectivity of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states; and (2) a C₂₊ yield that is increased by equal to or greater than about 5%, when compared to a C₂₊ yield of an otherwise similar OCM catalyst composition that is not characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

13. A method of making an oxidative coupling of methane (OCM) catalyst composition comprising:
(a) forming an oxide precursor mixture, wherein the oxide precursor mixture comprises one or more compounds comprising a Sr cation, one or more compounds comprising a Ce cation, and one or more compounds comprising a Yb cation, and wherein the oxide precursor mixture is characterized by a molar ratio of Sr:(Ce+Yb) that is not about 1:1; and
(b) calcining at least a portion of the oxide precursor mixture to form the OCM catalyst composition, wherein the OCM catalyst composition comprises Sr—Ce—Yb—O perovskite in an amount of less than about 75.0 wt. %.

14. The method of claim 13, wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states.

15. The method of claim 13, wherein the step (a) of forming an oxide precursor mixture further comprises (i) solubilizing the one or more compounds comprising a Sr cation, one or more compounds comprising a Ce cation, and one or more compounds comprising a Yb cation in an aqueous medium to form an oxide precursor aqueous solution; and (ii) drying at least a portion of the oxide precursor aqueous solution to form the oxide precursor mixture.

16. The method of claim 15, wherein the oxide precursor aqueous solution is dried at a temperature of equal to or greater than about 75° C.

17. The method of claim 13, wherein the oxide precursor mixture is calcined at a temperature of equal to or greater than about 650° C.

18. The method of claim 13, wherein the one or more compounds comprising a Sr cation comprises Sr nitrate, Sr oxide, Sr hydroxide, Sr chloride, Sr acetate, Sr carbonate, or combinations thereof; wherein the one or more compounds comprising a Ce cation comprises Ce nitrate, Ce oxide, Ce hydroxide, Ce chloride, Ce acetate, Ce carbonate, or combinations thereof; and wherein the one or more compounds comprising a Yb cation comprises Yb nitrate, Yb oxide, Yb hydroxide, Yb chloride, Yb acetate, Yb carbonate, or combinations thereof.

19. An OCM catalyst produced by the method of claim 13.

20. A method for producing olefins comprising:
(a) introducing a reactant mixture to a reactor comprising an oxidative coupling of methane (OCM) catalyst composition, wherein the reactant mixture comprises methane (CH₄) and oxygen (O₂), wherein the OCM catalyst composition is characterized by the overall general formula Sr_{1.0}CeₐYb_{b}O_{c}, wherein a is from about 0.01 to about 2.0, wherein b is from about 0.01 to about 2.0, wherein the sum (a+b) is not 1.0, and wherein c balances the oxidation states;
(b) allowing at least a portion of the reactant mixture to contact at least a portion of the OCM catalyst composition and react via an OCM reaction to form a product mixture comprising olefins;
(c) recovering at least a portion of the product mixture from the reactor; and
(d) recovering at least a portion of the olefins from the product mixture.
